(19) United States
(12) Patent Application Publication
(10) Pub. No.: US 2016/0228664 A1
(43) Pub. Date: Aug. 11, 2016

(54) METHOD TO DECREASE BRAIN INJURY FOLLOWING CEREBRAL ISCHEMIA

(71) Applicant: Joel Steven Goldberg, Hillsborough, NC (US)

(72) Inventor: Joel Steven Goldberg, Hillsborough, NC (US)

(21) Appl. No.: 15/131,665

(22) Filed: Apr. 18, 2016

(51) Int. Cl. A61M 16/06; A61M 16/00; A61M 16/20

(52) U.S. Cl. CPC A61M 16/06; A61M 16/208; A61M 16/0057; A61M 2202/0208

(57) ABSTRACT

A method to immediately treat cerebral ischemia caused by hemorrhage, thrombosis or spasm is described. The method requires an apparatus comprising a tight fitting non-rebreathing mask with one way expiratory valves and a pressure regulated source of compressed oxygen. The mask may have an optional port for the administration of nitric oxide. Use of this apparatus can oxygenate and denitrogenate ischemic brain tissue and decrease infarct size. Little training is required for proper use of the apparatus which can be purchased for less than 75 USD in the United States. Similar to a fire extinguisher, this apparatus can be stored in the home and at work available for emergency use especially for those at high risk for stroke. A clinical trial should be conducted to assure that this therapy does no harm regardless of the etiology of cerebral ischemia. The cost of such a trial is not expected to be prohibitive and the institutional review board overseeing such a study should recognize the expected low risk to benefit ratio.

### CROSS-REFERENCES TO RELATED APPLICATIONS

None

### FEDERALLY FUNDED RESEARCH

Not applicable

### BACKGROUND OF THE INVENTION

Commonly referred to as a stroke, brain injury from cerebral ischemia is a devastating unpredictable occurrence. The neural deficits associated with stroke are major causes of global morbidity and mortality.

Three major causes of stroke are hemorrhage, thrombosis and spasm of a cerebral artery. Hemorrhage may be prevented by regulation of blood pressure. Thrombosis may be prevented by anticoagulation which is often prescribed after artificial valve replacement or onset of atrial fibrillation, carotid artery dissection, left ventricular mural thrombi or right to left intracardiac shunt. Spasm after subarachnoid bleeding, migraine headache and trauma may be treated with vasodilators and therapies of hemodilution, hypertension and hypervolemia.

When a portion of the brain becomes ischemic, there is a critical time interval to institute therapy for preservation of brain tissue. Common therapies include antithrombolytic agents, systemic vasodilators or vasoconstrictors that can regulate blood pressure and reverse cerebral vasospasm. Oxygen and anticonvulsant medication are usually administered.

When a stroke occurs, the history and physical examination of the patient may not provide enough information to determine the etiology of the stroke. In such instances, imaging at a medical center is required to diagnose if the stroke is secondary to hemorrhage, thrombosis or spasm. Pharmacologic therapies are determined by the underlying etiology of the stroke. There is need for a treatment that precedes determination of stroke etiology and can be performed at home or work prior to presentation to a medical center. This therapy can be initiated with little training and is low risk.

There are four gases known to have physiologic effects in the human body. These gases are oxygen, nitrogen, carbon dioxide and nitric oxide. Oxygen is required for oxidative phosphorylation and production of ATP. Nitrogen is required to dilute oxygen and prevent oxygen toxicity. Carbon dioxide is produced during the metabolism of pyruvate in the citric acid cycle. Nitric oxide is a signaling agent and is administered to treat pulmonary hypertension. Its role during a cerebral ischemic event is well not understood.

During and following an ischemic event to the brain, oxygen therapy is commonly administered. Animal models suggest that normobaric oxygen therapy effectively slows cerebral ischemia as measured by damage to the blood brain barrier. (Liang et al., 2015) High flow normobaric oxygen therapy has been shown to transiently reduce cerebral infarct size as measured by MRI when administered within 12 hours of the ischemic event.(Singhal et al., 2005) This is in contrast to a study that found no improvement in clinical scores of stroke when high flow oxygen was administered within 8 hours after the ischemic event.(Padma et al., 2010) Long term outcomes were not significantly different in either study. These studies used high flow oxygen with a simple face mask and did not include specific treatments designed to denitrogenate the patients. Oxygen therapy without denitrogenation for the treatment of stroke may not be sufficient for improving clinical outcome because nitrogen may displace utilized oxygen and poison the mitochondria of cerebral tissue.(VanDeripe, 2004) (FIG. 1) Therefore, oxygenation with substantial denitrogenation is most likely to improve clinical outcome, during and after a stroke.

Heliox, a mixture of oxygen and helium, has been shown in an animal model to decrease ischemic induced injury when compared to administration of 100% oxygen and a control group.(Pan, Zhang, VanDeripe, Cruz-Flores, & Panneton, 2007) Furthermore the effect of heliox reduction on infarct size in an animal model was greatest when the gas was administered immediately after ischemia. Although heliox may have some advantages compared with 100% oxygen therapy, the gas is expensive and unlikely to be available for rare emergencies at home or work. In addition the oxygen content of the heliox mixture may not be sufficient to treat cerebral ischemia especially in patients who suffer concomitant pulmonary or cardiac disease.

Oxygen is commonly stored in pressurized tanks that can be regulated for use. Denitrogenation requires a specialized tight fitting non-rebreathing mask with one way expiratory flow valves. This invention describes an immediate cost effective method to oxygenate and denitrogenate a patient during and following a stroke regardless of etiology and can be performed outside a medical center such as at home or work. This invention also provides a human model supporting proof of concept that denitrogenation in addition to oxygenation is preferred for treatment of an ischemic limb where the results could be extended to the treatment of cerebral ischemia.

Prior art of established therapies utilizing normobaric oxygen and denitrogenation:

| | Diagnosis | Gas | Setting |
|---|---|---|---|
| Noninvasive treatment of | Pneumo- | 100% oxygen | In hospital |
| pneumothorax with | thorax | | |
| oxygen inhalation. | | | |
| Respiration. | | | |
| 1983; 44(2): 147-52. | | | |
| Chadha TS, Cohn MA. | | | |
| U.S. Pat. No. 7,263,993 B2 | Cerebral | Helium and | Ambulance |
| | ischemia | oxygen | and in |
| | | | hospital |
| Venous Air Embolism | Air | 100% oxygen | In hospital |
| Arch Intern Med. | embolism | | |
| 1982; 142(12): 2173-2176. | | | |
| Ronald J. O'Quin, MD; | | | |
| S. Lakshminarayan, MD | | | |
| Hyperbaric or normobaric | Carbon | 100% oxygen | In hospital |
| oxygen for acute carbon | monoxide | | |
| monoxide poisoning: a | poisoning | | |
| randomized controlled | | | |
| clinical trial. Med J Aust. | | | |
| 1999 Mar 1; 170: 203-10. | | | |
| Scheinkestel CD, Bailey M, | | | |
| Myles PS, et al. | | | |
| High-flow oxygen therapy | Respiratory | 100% oxygen | In hospital |
| in acute respiratory | failure | | |
| failure. Respiratory Care | | | |
| 55.4 (2010): 408-413. | | | |
| Roca, Oriol, et al. | | | |

### DESRCIPTION OF THE DRAWING

FIG. 1 shows that removal of nitrogen is required to resuscitate ischemic brain tissue. Label 1 shows nitrogen microbubbles, label 2 shows oxygen microbubbles, label 3 shows carbon dioxide microbubbles, label 4 shows nitric oxide microbubbles, label 5 shows demarcation of an ischemic area in a cerebral artery.

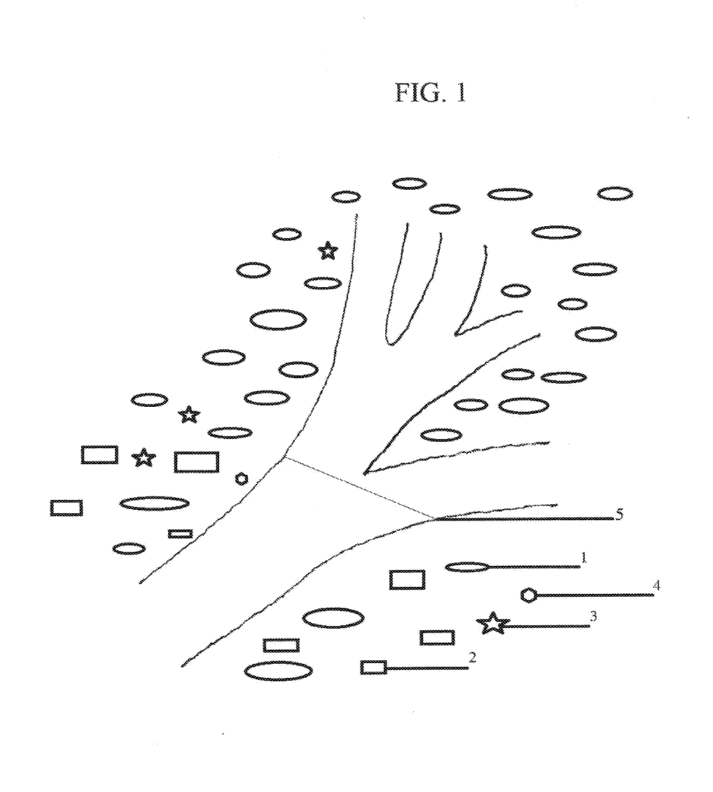

### DETAILED DESCRIPTION OF THE INVENTION

Instituted early, oxygenation and denitrogenation of ischemic brain tissue can improve clinical outcome for patients who have suffered a stroke regardless of cause. Oxygen can be administered from a regulated pressurized oxygen cylinder via a non-rebreathing mask. Denitrogenation can be accomplished using a non-breathing mask with an air tight fit and one way expiratory valves. An optional injection port can be constructed on the mask to facilitate nitric oxide administration if needed.

Clinical studies have shown that preoxygenation or denitrogenation of the lung can occur within 1 minute after a patient takes 8 deep breaths while fitted with a tight fitting mask with a one way expiratory valve, using high flow oxygen and an oxygen reservoir bag. (Tanoubi, Drolet, & Donati, 2009) Denitrogenation of the brain will take longer than denitrogenation of the functional residual capacity of the lung but will follow the brain to lung nitrogen concentration gradient. Complete denitrogenation of the brain is not required for the therapy described in this invention to be successful. However, enough nitrogen needs to be eliminated from the brain to permit oxygen to enter the mitochondria so that sufficient ATP is produced to maintain cell function.

In contrast to other therapies to treat cerebral ischemia, this invention requires that patients store oxygen and a non-rebreathing mask at home and at work for immediate use if stroke occurs. Unlike prior treatments for cerebral ischemia, the therapy described in this invention is not dependent upon the etiology of the stroke. Also oxygenation and denitrogenation therapy may be useful for the treatment of myocardial ischemia.(Goldberg, 2011)

### Experimental Section

In a human, pulse oximeters were attached to the left fourth digit and the right fourth digit. A blood pressure cuff was positioned on the left forearm and inflated to 130 mm Hg just below the systolic blood pressure. The hand slowly became ischemic with an O₂ saturation of 94% on the left digit compared with 99% on the right digit. 100% oxygen was then administered at 15 liters minute per minute through an air tight face mask via an anesthesia machine. Very shortly the O₂ saturation of the right digit was 100% and the left remained ischemic at 94%. After 2 minutes the oxygen saturation began to increase in the ischemic digit. After 8 minutes of ischemia and 6 minutes of 100% oxygen administration the O₂ saturation of the left digit was 98% and 100% on the right digit. The blood pressure cuff was deflated and room air was administered as the saturation in the two digits equilibrated to 99%. The results from this experiment strongly suggest that treatment of an ischemic digit requires denitrogenation in addition to oxygenation that serves as a model of cerebral ischemia.

### BENIFITS TO SOCIETY

Oxygenation and denitrogenation during and following a stroke can increase viable brain tissue after cerebral ischemia. The apparatus required to treat a stroke consists of a tight fitting non-rebreathing mask with one way expiratory valves and a regulated source of compressed oxygen. This apparatus can be stored in the home or at work for emergencies similar to a fire extinguisher. Regardless of stroke etiology, this treatment can be administered immediately. Minimal training is required to use the apparatus as a first line therapy. The apparatus can be obtained in the US at a cost of less than 75 USD. A clinical outcome trial should be conducted to assure that this therapy does no harm. The cost of such a trial is not expected to be prohibitive and the institutional review board overseeing such a study should recognize the expected low risk-benefit ratio. Also oxygenation and denitrogenation therapy may be useful for the treatment of myocardial ischemia.

### REFERENCES

Goldberg, Joel S. (2011). Atherosclerosis: Viewing the Problem from a Different Perspective Including Possible Treatment Options. Lipid Insights, 4, 17-26.
Liang, J., Qi, Z., Liu, W., Wang, P., Shi, W., Dong, W., . . . Liu, K. J. (2015). Normobaric hyperoxia slows blood-brain barrier damage and expands the therapeutic time window for tissue-type plasminogen activator treatment in cerebral ischemia. Stroke, 46(5), 1344-1351. doi: 10.1161/STROKEAHA.114.008599
Padma, M. V., Bhasin, A., Bhatia, R., Garg, A., Singh, M. B., Tripathi, M., & Prasad, K. (2010). Normobaric oxygen therapy in acute ischemic stroke: A pilot study in Indian patients. Ann Indian Acad Neurol, 13(4), 284-288. doi: 10.4103/0972-2327.74203
Pan, Y., Zhang, H., VanDeripe, D. R., Cruz-Flores, S., & Panneton, W. M. (2007). Heliox and oxygen reduce infarct volume in a rat model of focal ischemia. Exp Neurol, 205(2), 587-590. doi: 10.1016/j.expneuro1.2007.03.023
Singhal, A. B., Benner, T., Roccatagliata, L., Koroshetz, W. J., Schaefer, P. W., Lo, E. H., . . . Sorensen, A. G. (2005). A pilot study of normobaric oxygen therapy in acute ischemic stroke. Stroke, 36(4), 797-802. doi: 10.1161/01.STR.0000158914.66827.2e
Tanoubi, I., Drolet, P., & Donati, F. (2009). Optimizing preoxygenation in adults. Can J Anaesth, 56(6), 449-466. doi: 10.1007/s 12630-009-9084-z
VanDeripe, D. R. (2004). The swelling of mitochondria from nitrogen gas; a possible cause of reperfusion damage. Med Hypotheses, 62(2), 294-296. doi: 10.1016/S0306-9877(03)00304-9

Having described my invention, I claim:

1. A method to immediately treat cerebral ischemia that occurs outside a medical center with an apparatus comprising a non-rebreathing face mask with one way expiratory valves and a regulated source of oxygen.

2. The method in claim 1 that immediately treats cerebral ischemia secondary to hemorrhage, thrombosis or spasm.
